(19) United States
(12) Patent Application Publication
(10) Pub. No.: US 2002/0132855 A1
(43) Pub. Date: Sep. 19, 2002

(54) Use of acetaminophen to prevent and treat arteriosclerosis

(76) Inventors: Edward B. Nelson, Lower Gwynedd, PA (US); Charles V. Smith, Blacklick, OH (US); Addison A. Taylor, Houston, TX (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/887,465

(22) Filed: Jun. 22, 2001

Related U.S. Application Data
(60) Provisional application No. 60/222,781, filed on Aug. 3, 2000.

(51) Int. Cl. A61K 31/455; A61K 31/375; A61K 31/366; A61K 31/401; A61K 31/355; A61K 35/78; A61K 31/165

(52) U.S. Cl. 514/629; 514/356; 514/458; 514/474; 424/757; 514/423; 514/460; 514/547

(57) ABSTRACT

This invention relates to a method of treating, preventing, and regressing arteriosclerosis including atherosclerosis in mammals by administering an effective amount of acetaminophen or pharmaceutically acceptable salt thereof and optionally a second active ingredient, along with compositions containing the same.

### CROSS-REFERENCE TO RELATED APPLICATION

This Application claims the benefit of U.S. application Ser. No. 60/222,781 filed on Aug. 3, 2000, which is incorporated by reference in its entirety herein.

### FIELD OF THE INVENTION

This invention relates to a method for preventing, treating, and regressing arteriosclerosis, and in particular atherosclerosis, via the administration of an effective amount of acetaminophen.

### BACKGROUND OF THE INVENTION

Arteriosclerosis is understood to be a pathologic condition of thickening and loss of elasticity of arterial walls. See Dorland's Illustrated Medical Dictionary, p. 143 (25^{th} Ed. 1974). One form of arteriosclerosis is atherosclerosis, which is understood to be a pathologic condition of degeneration and hardening of the walls of arteries and sometimes the valves of the heart. Atherosclerosis is also associated with an accumulation of fatty lipid oxidation products in the walls of such blood vessels. See Dorland's Pocket Medical Dictionary, p. 74 (21^{st} Ed. 1968).

Acetaminophen, N-(4-hydroxyphenyl) acetamide, also known as paracetamol, or herein referred to as APAP, was first used in medicine by Van Mering in 1893, but only since 1949 has it gained in popularity as an effective alternative to aspirin for analgesic uses in the over the counter market. The pharmacology of APAP is reviewed by B. Ameer et al., Ann. Int. Med. 87, 202 (1977) and the preparation of APAP is disclosed in U.S. Pat. No. 2,998,450. Considering the widespread use of APAP and the volume of its manufacture, both its manufacture and its use as an analgesic are well known to persons skilled in the art.

Unlike aspirin, which decreases platelet aggregation, APAP has not been found to produce any in vivo cardiovascular benefits. As disclosed in Nensetter, “Paracetamol Inhibits Copper Ion-induced Azo Compound-initiated and Mononuclear Cell-Mediated Oxidative Modification of LDL,” 115(9) Arterio. Thromb. Vasc Biol. 1338(1995), APAP has been shown to prevent low density lipoprotein (“LDL”) lipid oxidation in vitro. Although the prevention of LDL lipid oxidation may be a factor in the prevention of atherosclerosis, it is apparent that other factors are also necessary for successful atherosclerosis prevention. For example, the analogs and metabolites of probucol, which is known for potently preventing LDL oxidation, were not effective in the treatment and prevention of atherosclerosis. See Fruebis, et al., “A Comparison of the Antiatherogenic Effects of Probucol and of a Structural Analogue of Probucol in Low Density Lipoprotein Receptor-Deficient Rabbits,” 94 J. Clin. Invest. 392-98 (1994)(analog) and Witting, et al., “Dissociation of Atherogenesis From Aortic Accumulation of Lipid Hydro(pero)xides in Watanabe Heritable Hyperlipidemic Rabbits”, 104 J. Clin. Invest. 213-220 (1999)(metabolite).

It is well-known that atherosclerosis may be treated by surgical means to open or bypass narrowed coronary vessels. Pharmacologic treatment for atherosclerosis is generally limited to lowering total blood cholesterol levels via following a low fat/low cholesterol diet and/or taking certain drugs such as statins or phytosterols on a regular basis. While these treatments are somewhat effective, there remains a continuing need for additional treatments for this disease, and perhaps a regimen that could induce the regression of and/or prevent the occurrence of atherosclerosis.

### SUMMARY OF THE INVENTION

This invention relates to a method of treating, regressing, and/or preventing arteriosclerosis, and in particular atherosclerosis, in a patient through the administration of an effective dose comprising, consisting of, and/or consisting essentially of APAP, or pharmaceutically acceptable salts, isomers, esters, ethers and/or prodrugs thereof. In an alternative embodiment, an effective amount of a second active ingredient for treating, regressing, and/or preventing atherosclerosis, arteriosclerosis, or coronary disease such as anti-platelet agents, sitosterols, sitostanols and/or statins are employed.

Another embodiment of the present invention is directed to a composition comprised, consisting of, and/or consisting essentially of of APAP and a secondary agent for the treatment, regression, and/or prevention of arteriosclerosis, and in particular atherosclerosis.

### DESCRIPTION OF THE DRAWINGS

FIG. 1 depicts the spectrometric absorbance at 234 nm of isolated LDL versus time (hours) as determined via Dienes analysis.

### DETAILED DESCRIPTION OF THE INVENTION

This invention relates to a method for treating, regressing and/or preventing arteriosclerosis, and in particular atherosclerosis, in mammals using an effective amount of a primary agent selected from APAP and pharmaceutically acceptable salts, isomers, esters, ethers and/or prodrugs thereof, and compositions for the same. As used herein, “effective amount” shall mean an amount that is sufficient to provide prevention, regression, and/or treatment of arteriosclerosis, and in particular atherosclerosis. “Mammals,” as used herein, shall mean any of a class of warm-blooded higher vertebrates that nourish their young with milk secreted by mammary glands and includes humans and animals.

As used herein, “treatment”, is meant to stop the progression, and if possible induce a regression, of the fatty layer accumulations along blood vessel walls of a patient containing such accumulations. As used herein, “prevention” is meant to proactively stop the development of arteriosclerotic lesions, e.g. fatty streaks. As used herein, “regression” is meant to reduce the amount of fatty layer accumulations along blood vessel walls of a patient containing such accumulations.

By effectively treating, preventing, and/or regressing the development of such fatty layer accumulations, the method of the present invention will lead to the prevention and/or treatment of further conditions associated with atherosclerosis including, but not limited to low-flow ischemia, angina pectoris, coronary vasoplasm, congestive heart failure, mycocardial ischemia, heart attack, stroke, and for various uses in cardiothoracic surgery including cardioplegia, and angioplasty.

The present invention contemplates the treatment of a human or animal, through So any suitable dosage forms. Suitable forms include solids, such as tablets or powders, liquids including suspensions, dispersions and solutions and the like. The preferred route of administration of the APAP is via oral administration, although other techniques, such as transdermal delivery, known in the art may also be employed.

Examples of suitable pharmaceutically acceptable salts of acetaminophen include inorganic salts of acetaminophen, including: sodium, calcium, lithium, potassium, magnesium, cesium, ammonia, ferrous, zinc, manganous, aluminum, ferric, manganic, and the like, organic salts of acetaminophen with primary, secondary, tertiary and quaternary amines, substituted amines including naturally occurring substituted amines, cyclic amines and basic ion exchange resins, triethylamine, tripropylamine, 2-dimethylaminoethanol, 2-diethylaminoethanol, lysine, arginine, histidine, caffeine, procain, N-ethylpiperidine, hydrabamine, choline, betaine, ethylenediamine, glucosamine, methylglycamine, theobromine, pruines, piperazine, piperidine, polyamine resins and mixtures thereof.

Examples of suitable prodrugs of APAP include propacetamol.

Examples of suitable ethers of APAP include phenacetin.

The effective dosage range of APAP is from about 10 mg to less than or equal to about 4000 mg, in particular about 25 mg to about 1000 mg or about 200 mg to about 500 mg at intervals of about 1 to about 4 times per day for an average (70 KG) human, e.g. from about 0.14 mg/kg to less than or equal to about 57 mg/kg.

One skilled in the art would readily appreciate without undue experimentation that the typical doses of the pharmaceutically acceptable salts, prodrugs, ethers, and isomers of APAP will vary according to the molecular weight of the salt, prodrug, ether, and isomer, respectively, required to give the dose of APAP equivalent to that set forth above. Typically doses are taken every four to six hours, but care should be taken to avoid exceeding the daily maximum recommended dosage of acetaminophen of 4000 milligrams per day.

In one embodiment, a continuous low level of APAP or pharmaceutically acceptable salt thereof, e.g. about 10 mg to about 4000 mg of total APAP, may be maintained in the body at all times or at least from about 4 hours to about 12 hours per day. For example, the required dosage of APAP may be administered via a tablet having a sustained release coating such as that disclosed in U.S. Pat. Nos. 6,210,714, 5,773,031, and 5,004,613, which are incorporated by reference herein.

The present invention can incorporate a secondary agent for the treatment of arteriosclerosis, and in particular atherosclerosis. Specific groups, of secondary agents which may be used to treat arteriosclerosis or coronary disease (a consequence of arteriosclerosis) nonexclusively include cholesterol-lowering agents, e.g. low density lipoprotein (“LDL”) lowering agents or high density lipid (“HDL”) raising agents, antioxidants, antiplatelet agents, cholesterol-absorption inhibitors, cholesterol synthesis inhibitors, and mixtures thereof.

Examples of suitable cholesterol-lowering agents include, but are not limited to, statins, fibrates, niacin, and mixtures thereof.

In particular, suitable statins include, but are not limited to atorvastatin, cerivastatin, lovastatin, pravastatin, simvastatin, and mixtures thereof.

Examples of suitable antioxidant agents include, but are not limited to, vitamin E, vitamin C, and mixtures thereof.

Examples of antiplatelet agents include, but are not limited to aspirin, Glycoprotein IIaIIIb receptor inhibitors such as tirofiban hydrochloride available from Merck, Inc. under the tradename, “Aggrastat,” and mixtures thereof.

Examples of suitable cholesterol-absorption inhibitors include stanol fatty acid esters such as sitostanol fatty acid esters, sitosterol fatty acid esters, soy and derivatives thereof such as protein and isoflavones, and mixtures thereof.

Examples of suitable cholesterol synthesis inhibitors include, but are not limited to squaline oxidase inhibitors.

Preferred secondary agents include statins, sitostanols, sitosterols, aspirin, and mixtures thereof.

The amount of secondary agent to be used in the present invention will vary based upon the particular secondary agent selected. The effective dosing amounts for the secondary agent are well known in the art and are disclosed in, for example, The Physician's Desk Reference (2000), which is incorporated by reference herein. For example, during preparation of a combination pharmaceutical composition containing APAP and atorvastatin, the latter of which is commercially available from Pfizer, Inc. under the tradename “Lipitor,” the APAP is compounded with the atorvastatin in a suitable carrier in proportions of about 8/1 to about 1/400 parts by weight of atorvastatin or pharmaceutically acceptable salt thereof for each part by weight of APAP or pharmaceutically acceptable salt, isomer, ether, ester, and/or prodrug thereof.

To prepare the combination pharmaceutical compositions of the present invention, the compounds are intimately admixed with a pharmaceutical carrier according to conventional pharmaceutical compounding techniques, which carrier may take a wide variety of forms depending on the form of preparation desired for administration, e.g. oral or parenteral such as intra muscular. In preparing the compositions in oral dosage form, any of the usual pharmaceutical media may be employed. Thus, for liquid oral preparations, such as for example, suspensions, elixirs and solutions, suitable carriers and additives include solvents, suspending agents, sweeteners, flavoring agents, preservatives, coloring agents, and the like and mixtures thereof. See for example, U.S. Pat. No. 5,409,907, hereby incorporated by reference. For solid oral preparations such as, for example, powders, capsules, and tablets, suitable carriers and additives include compressible diluents, binders, disintegrants, wetting agents, glidants, lubricants, sweeteners, flavors, colors, and the like and mixtures thereof.

Suitable solvents include water, glycols, alcohols and the like and mixtures thereof.

Suitable suspending agents include methylcellulose, hydroxypropylmethylcellulose, hydroxyethylcellulose, carboxymethylcellulose, microcrystalline cellulose, xanthan gum, and the like, and mixtures thereof.

Suitable sweeteners include sugars such as glucose, fructose, sucrose, maltose; polyhedric alcohols such as sorbitol, mannitol, xylitol, maltitol, erythritol, and the like; high intensity sweeteners such as aspartame, saccharin, acesulfame-potassium, sucralose, and the like, and mixtures thereof.

Flavoring agents include peppermint, spearmint, cinnamon, vanilla, and the like, and mixtures thereof. A more complete listing of appropriate additives can be found in numerous publications including Remington's Encyclopedia.

Suitable compressible diluents for tablets, capsules, and powders include cellulose derivatives such as microcrystalline cellulose; compressible carbohydrates which include polyhedric alcohols such as mannitol, sorbitol, xylitol, maltitol, and the like, compressible sugars such as lactose, dextrose, sucrose, and the like; compressible inorganic salts such as calcium phosphate, calcium sulfate, calcium carbonate, magnesium hydroxide, magnesium carbonate, and the like, and mixtures thereof.

Suitable tablet binders include cellulose, cellulose derivatives, polyvinyl pyrrolidone, polyethylene glycol, starch, modified starch, gelatin, natural or synthetic gums such as xanthan gum, alginate, dextran, acacia gum, karaya gum, locust bean gum, tragacanth and the like, and mixtures thereof.

Suitable disintegrants, include starch, sodium starch glycolate, crosscarmellose, crosspovidone, microcrystalline cellulose and the like, and mixtures thereof.

Suitable wetting agents include glyceryl mono-stearate, polysorbates, lecithin, and the like, and mixtures thereof.

Suitable glidants include colloidal silicon dioxide and the like, and mixtures thereof.

Suitable lubricants include magnesium stearate, stearic acid, and the like, and mixtures thereof.

Suitable sweeteners include sugars such as glucose, fructose, sucrose, maltose; polyhedric alcohols such as sorbitol, mannitol, xylitol, maltitol, erythritol, and the like; high intensity sweeteners such as aspartame, saccharin, acesulfame-potassium, sucralose, and the like, and mixtures thereof.

Because of their ease in administration, tablets and capsules represent the most advantageous oral dosage unit form. In which case solid pharmaceutical carriers are obviously employed. If desired, tablets may be film coated, sugar coated, or enteric coated by standard techniques. For parenterals, the carrier will usually comprise sterile water, through other ingredients, for example, for purposes such as aiding solubility, osmolality, or for preservation, may be included. Injectable suspensions may also be prepared, in which case appropriate liquid carriers, suspending agents, and the like, and mixtures thereof may be employed.

The pharmaceutical compositions herein will contain, per dosage unit, e.g. tablet, capsule, powder, injection, teaspoonful and the like, an amount of the APAP to deliver an effective dose as described above. Alternatively, the compositions herein may contain a fraction of such a dose so that a variety of individual patients can closely match their dosage requirements with the administration of one, two or three tablets. Of course, instead of administering the active ingredients as a single composition, they may be administered simultaneously or sequentially as separate compositions.

Although the proportion by weight of active ingredient in the total dosage form will vary according to, for example, the compressibility of the active ingredient, and considerations such as convenient size of the dosage form, and the type of dosage form (for example, swallowable tablet, chewable tablet, or liquid), typically the active ingredients may comprise, based upon the total weight of the dosage form, from about 1% to about 95%, for example from about 5% to about 85%.

Another embodiment of the present invention is directed to a composition comprising APAP or pharmaceutically acceptable salt, isomer, ester, ether, and/or prodrug thereof and one or more of the secondary agents in amounts set forth above.

In order to illustrate the invention, the following examples are included. These examples do not limit the invention. They are meant only to suggest a method of practicing the invention.

### EXAMPLES

The following test method was used in the Examples:

LDL Isolation: Plasma LDL was prepared by ultracentrifuation of a blood sample as disclosed in Havel, “The Distribution and Chemical Composition of Ultracentrifugally Separated Lpoproteins in Human Serum,” 34 JI. Clin. Inv. 1345 (1995), within a density cutoff of 1.020 g/ml to 1.055 g/ml and with the addition of 0.5 mg/ml ethylenediaminetetracetic acid (‘EDTA’) thereto. After adding a sample of this mixture to a semi-permable bag, the LDL was dialyzed therefrom against a vacuum-degassed 0.01 M Tris solution containing 0.15 M NaCl and 0.1 mg/ml EDTA under dark conditions and at 4° C. for 48 hours, followed by dialysis overnight using the same equipment but with a 0.01M phosphate buffer solution containing 0.15 M NaCl and no EDTA. The dialyzed LDL protein concentrations were then determined via the method set forth in Lowry, ‘Protein Measurement with the Folin Phenol Reagent,’ 226 J. Biol. Chem. 497-509 (1951).

### Example 1.

This investigation used an experimental system in which fatty streaks are induced in the blood vessels of rabbits by incorporation of 1 % cholesterol in their food. For further detail, see A. A. Taylor and C. V. Smith, Circulation 18 I-697 (1999). Similar experimental systems have been used previously for the evaluation of the efficacy of pharmaceutical compounds for treating, preventing, and regressing atherosclerosis. For example, see Daugherty, A., et al., “Probucol Attentuates the Development of Aortic Atherosclerosis in Cholesterol-Fed Rabbits,” 98(2) Br. J. Pharm. 612-18 (1989), which is incorporated by reference herein.

Twenty-three, six-week old male New Zealand white rabbits obtained from Myrtle's Rabbitry (Houston, Tex.) and weighing between about 1.8 kg and 2 kg were separated into two groups: Group 1 (experimental) having 12 rabbit/group and Group 2 (control) having 11 rabbits/group. At the beginning of the study, all rabbits were allowed water ad libitum and ate normal rabbit chow.

After a two week acclimatization period, all of the rabbits were then placed on normal rabbit chow supplemented with 1% cholesterol (ICN Biochemicals, Inc., Costa Mesa, Calif.), in order to induce fatty streak formation in their blood vessel walls. Rabbits in Group 1 were switched from water to an acetaminophen solution for about a 40 mg/kg_{rabbit} APAP dose. A 40 mg/kg dose of APAP in a 2.5 kg rabbit is equivalent to about a 2800 mg/day dose in a 70 kg human. The other group of rabbits remained on water for the remainder of the study. The cages were monitored daily in order to assure that each rabbit consumed the contents of his respective water bottle.

After 12 weeks, each rabbit was anesthetized with a cocktail of ketamine/acepromazine/xylazine (42.8 mg/ml:8.2 mg/ml:1.4 mg/ml) in an amount of 0.1 ml cocktail/kg of rabbit. After removing the aorta of each rabbit from the aortic annulus to the iliac bifurcation and opening the same longitudinally, the removed aorta section was pinned to a silicone plate and photographed with a blue filtered light using a digital camera available from Olympus. After enlarging the digital images, the total aortic area and the area comprised of fatty streak was blindly measured by planimetry using software available from Jandel Scientific under the tradename, “SigmaScan.” The results are shown below in Table B:

TABLE A
Fatty Streak/Total Aortic Area Ratio

| Rabbits Tested | Average Fatty Streak/Total Aortic Area |
|---|---|
| APAP - fed | 0.32 + 0.05 |
| Control | 0.58 + 0.06 |

This analysis showed that the administration of APAP produced significantly reduced aortic fatty streak areas (p<0.001), which is well known as an initial sign of atherosclerosis. See, for example, Weissberg, “Coronary Disease-Atherogenesis: Current Understanding of the Causes of Atheroma,” Heart 2000;83:247-252.

Oxo-sterol concentrations in the isolated LDL were also determined by the method set forth in Hughes, et. al., “Cytotoxicity of Oxidized LDL to Porcine Aortic Smooth Muscle Cells Is Associated with the Oxysterols 7-ketocholesterol and 7-hydrocholesterol,” 14(7) Arterioscler. Thromb. 1177-85 (1994). In general, the oxysterols were initially separated from the LDL via a Bligh and Dyer extraction followed by extraction using amino-propyl solid phase extraction columns available from Varian Instruments in order to further separate the lipids by polarity. The oxysterol-containing eluents were then dried therefrom under a nitrogen atmosphere and derivatized with N,O-bis(trimethylsilyl)trifluooroacetamide) (“BSTFA”) in a test tube. 10 μl of cholestane was added to the dried eluent at the time of derivatization as an internal standard. The derivatized samples were then analyzed using a gas chromatograph column having a flame ionization detector .The column program included initial temperature of 250° C. for 2 minutes, followed by temperature increases of 8° C./minute to 325° C., and then a final 8 minute temperature hold at 325° C. The amount of oxysterols in the LDL were then quantitated using known standards by ratio of the peak area of the oxysterol to cholestane. The concentrations of oxysterols are set forth below in Table B:

TABLE B
Concentration of Oxysterols (nmol/mg LDL)

| Group | Average | Average | Average | Average | Average |
|---|---|---|---|---|---|
| Tested | 7aOH-cholesterol | 7bOH-cholesterol | 7keto-cholesterol | 5a, 6a-epoxide | 5b, 6b-epoxide |
| APAP-fed | 0.58 + 0.12 | 0.32 + 0.07 | 0.41 + 0.14 | 0.11 + 0.05 | 0.12 + 0.13 |
| Control | 2.31 + 0.62 | 1.01 + 0.26 | 3.86 + 1.72 | 0.49 + 0.08 | 0.24 + 0.05 |

This analysis indicated that the administration of APAP produced a 4 fold decrease in 7-alpha hydroxycholesterol values and a 3 fold decrease in 7-beta hydroxycholesterol values, and a 9-fold decrease in 7-keto cholesterol values. These findings suggest that APAP in doses therapeutically relevant to humans has anti-atherosclerotic properties. Without wishing to be bound by theory, one reason for this effect may be as a result of APAP's inhibitory effect on the formation of highly atherogenic oxysterols in LDL.

After cannulating the femoral artery and advancing into the distal aorta region of each rabbit, blood was aspirated therefrom for isolation of LDL fraction. The blood of each rabbit was drawn into 1000 U of Heparin sodium in order to prevent coagulation, then the plasma was separated therefrom via centrifugation at 2000 rpm for ten minutes. The plasma was either stored directly at −80° C. or prepared for subsequent LDL isolation in accordance with the test method set forth above. The results of this test are shown below in Table C.

TABLE C
Total Plasma Cholesterol Levels

| | Average Circulating total Plasma |
|---|---|
| Rabbits Tested | Cholesterol Level (mg/dl) |
| APAP - fed | 1592 ± 43 |
| Control | 1441 ± 87 |

This analysis showed that APAP did not produce a significant difference in the amount of circulating cholesterol in the blood plasma. The result that APAP produced significantly reduced aortic fatty streak areas without lowering cholesterol was unexpected.

In addition, 0.1 mg/ml of isolated LDL from each rabbit was added to a total volume of 12 ml of Ham's F-10 media and incubated with 5 μM CuSO₄ at a temperature of 37° C. for a total 24 hours for subsequent dienes and thiobarbituric acid reactive substances (“TBARS”) analysis, which are two of many known measurements of lipoprotein oxidation.

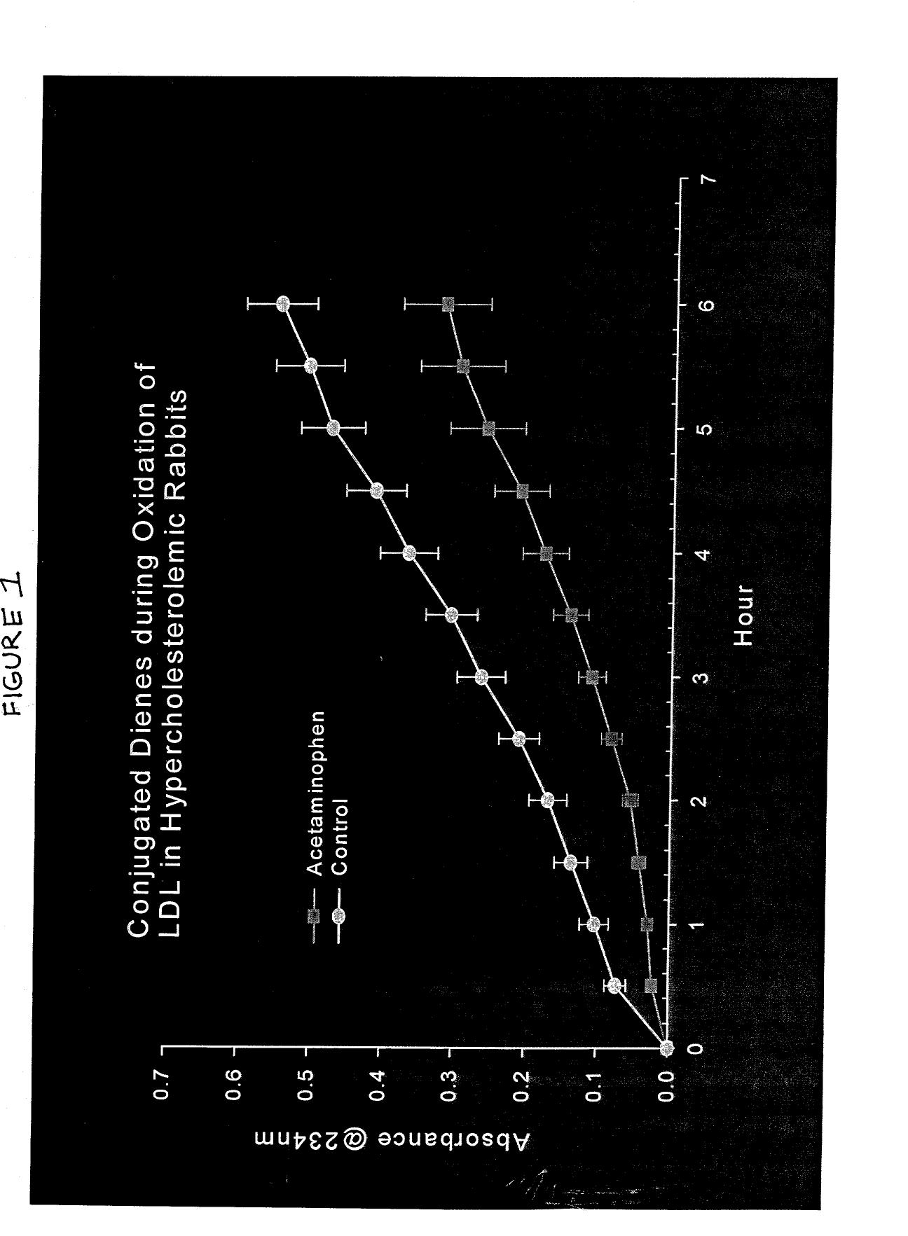

Dienes Analysis: The kinetics of these copper-mediated oxidation reactions were followed at 10 minute intervals for a total period of 6 hours by monitoring changes in absorbance at 234 nm of each mixture using a Model UV-2101PC spectrophotometer available from Shimadzu. For further details, See Esterbauer, et al., “Continuous Monitoring of In Vitro Oxidation of Human Low Density Lipoprotein,” 6 Free Radical Res. Comm. 67-75 to (1989). The spectrophotometric results shown in FIG. 1 revealed that the APAP-fed rabbits reported lower absorbance values over the 6 hour period. It was evident from this analysis indicates that APAP was an effective LDL anti-oxidant, and thus would be effective in the treatment, regression, and prevention of arteriogenicity and in particular, atherogenicity.

TBARS Analysis: At period of 4 hours, 8 hours, and 12 hours, the copper-mediated oxidation reactions were stopped by the addition of 10 μl of a 10% BHT per 2 ml of the copper-mediated oxidation reaction mixture, and two ml aliquots were respectively removed therefrom. The concentrations of thiobarbituric acid reactive substances (“TBARS”) in each mixture were then measured with the same spectrophotometer as described by Streinbrecher, et al., “Decrease in reactive amino groups during oxidation or endothelial cell modification of LDL,” Arteriosclerosis 1987;7:135-143. This Example indicated that the TBARS formed during the LDL oxidation were comparable in the two groups. However, in view of the fact that the dienes analysis showed the significant antioxidant activity of APAP, this comparable TBARS result obtained from the isolated LDL of the control and APAP-fed rabbits suggested that the copper sulfate may not have been very reactive with the thiobarbituric acid. Therefore, it is our belief that this TBARS assay did not specifically reflect the ongoing oxidation in the system.

What is claimed is:

1. A method for treating arteriosclerosis in a mammal comprising administering an effective dose of one or more of the following primary agents to a mammal in need thereof:
a) acetaminophen;
b) a pharmaceutically acceptable salt of acetaminophen;
c) a pharmaceutically acceptable isomer of acetaminophen;
d) a pharmaceutically acceptable ester of acetaminophen;
e) a pharmaceutically acceptable ether of acetaminophen;
f) a prodrug of acetaminophen; or
g) mixtures thereof.

2. The method of claim 1 wherein the amount of acetaminophen in the dose is greater than or equal to about 0.14 mg/kg of body mass of the mammal and is less than or equal to about 57 mg/kg of body mass of the mammal.

3. The method of claim 2, wherein the dose further comprises an effective amount of a secondary agent for the treatment of arteriosclerosis or coronary disease selected from the group consisting of cholesterol lowering agents, antioxidants, antiplatelet agents, cholesterol-absorption inhibitors, and mixtures thereof.

4. The method of claim 3 wherein the cholesterol-lowering agents are selected from the group consisting of statins, filtrates, niacin, and mixtures thereof.

5. The method of claim 3 wherein the antioxidant agents are selected from the group consisting of vitamin E, vitamin C, and mixtures thereof.

6. The method of claim 3 wherein the antiplatelet agents are selected from aspirin, IIaIIIb inhibitors, and mixtures thereof.

7. The method of claim 3 wherein the cholesterol-absorption inhibitors include stanol fatty acid esters, soy, and derivatives and mixtures thereof.

8. The method of claim 3 wherein the second active ingredient is a statin.

9. The method of claim 3 wherein the second active ingredient is atorvastatin.

10. A method for treating atherosclerosis in a mammal comprising administering an effective dose of one or more of the following primary agents to a mammal in need thereof:
a) acetaminophen;
b) a pharmaceutically acceptable salt of acetaminophen;
c) a pharmaceutically acceptable isomer of acetaminophen;
d) a pharmaceutically acceptable ester of acetaminophen;
e) a pharmaceutically acceptable ether of acetaminophen;
f) a prodrug of acetaminophen; or
g) mixtures thereof.

11. The method of claim 10 wherein the amount of acetaminophen in the dose is greater than or equal to about 0.14 mg/kg of body mass of the mammal and is less than or equal to about 57 mg/kg of body mass of the mammal.

12. The method of claim 10, wherein the dose further comprises an effective amount of a secondary agent for the treatment of atherosclerosis selected from the group consisting of cholesterol lowering agents, antioxidants, antiplatelets, cholesterol-absorption inhibitors, and mixtures thereof.

13. The method of claim 12 wherein the cholesterol lowering agents are selected from the group consisting of statins, filtrates, niacin, and mixtures thereof.

14. The method of claim 12 wherein the antioxidant agents are selected from the group consisting of vitamin E, vitamin C, and mixtures thereof.

15. The method of claim 12 wherein the antiplatelet agents are selected from the group consisting of aspirin, IIaIIIb inhibitors, and mixtures thereof.

16. The method of claim 12 wherein the cholesterol-absorption inhibitors are selected from the group consisting of stanol fatty acid esters, soy, and derivatives and mixtures thereof.

17. The method of claim 12 wherein the secondary agent is a statin.

18. The method of claim 12 wherein the secondary agent is atorvastatin.

19. A composition comprising:
A) one or more of the following primary agents:
1) acetaminophen;
2) a pharmaceutically acceptable salt of acetaminophen;
3) a pharmaceutically acceptable isomer of acetaminophen;
4) a pharmaceutically acceptable ester of acetaminophen;
5) a pharmaceutically acceptable ether of acetaminophen;
6) a prodrug of acetaminophen; or
7) mixtures thereof; in an amount effective for treating arteriosclerosis; and
B) a secondary agent selected from the group consisting of statins, sitostanols, sitosterols, aspirin, and mixtures thereof.

20. The composition of claim 19 wherein the amount of acetaminophen in the composition is greater than or equal to about 0.14 mg/kg of body mass of the mammal and is less than or equal to about 57 mg/kg of body mass of the mammal.

21. A composition comprising:
A) one or more of the following primary agents:
1) acetaminophen;
2) a pharmaceutically acceptable salt of acetaminophen;
3) a pharmaceutically acceptable isomer of acetaminophen;
4) a pharmaceutically acceptable ester of acetaminophen;
5) a pharmaceutically acceptable ether of acetaminophen;
6) a prodrug of acetaminophen; or
7) mixtures thereof; in an amount effective for treating atherosclerosis and
B) a secondary agent selected from the group consisting of statins, sitostanols, sitosterols, aspirin, and mixtures thereof.

22. The composition of claim 21 wherein the amount of acetaminophen in the composition is greater than or equal toabout 0.14 mg/kg of body mass of the mammal and is less than or equal to about 57 mg/kg of body mass of the mammal.

23. A method for preventing atherosclerosis in a mammal comprising administering an effective dose of one or more of the following primary agents to a mammal in need thereof:
a) acetaminophen;
b) a pharmaceutically acceptable salt of acetaminophen;
c) a pharmaceutically acceptable isomer of acetaminophen;
d) a pharmaceutically acceptable ester of acetaminophen;
e) a pharmaceutically acceptable ether of acetaminophen;
f) a prodrug of acetaminophen; or
g) mixtures thereof.

24. The method of claim 23 wherein the amount of acetaminophen in the dose is greater than or equal to about 0.14 mg/kg of body mass of the mammal and is less than or equal to about 57 mg/kg of body mass of the mammal.

25. A method for regressing atherosclerosis in a mammal comprising administering an effective dose of one or more of the following primary agents to a mammal in need thereof:
a) acetaminophen;
b) a pharmaceutically acceptable salt of acetaminophen;
c) a pharmaceutically acceptable isomer of acetaminophen;
d) a pharmaceutically acceptable ester of acetaminophen;
e) a pharmaceutically acceptable ether of acetaminophen;
f) a prod rug of acetaminophen; or
g) mixtures thereof.

26. The method of claim 25 wherein the amount of acetaminophen in the dose is greater than or equal to about 0.14 mg/kg of body mass of the mammal and is less than or equal to about 57 kg/mg of body mass of the mammal.
